(19) United States
(12) Patent Application Publication
(10) Pub. No.: US 2023/0382739 A1
(43) Pub. Date: Nov. 30, 2023

(54) CHEMICAL METHODS FOR DIAMOND SURFACE FUNCTIONALIZATION

(71) Applicant: The Trustees of Princeton University, Princeton, NJ (US)

(72) Inventors: Nathalie de LEON, Princeton, NJ (US); Robert KNOWLES, Princeton, NJ (US); Lila RODGERS, Princeton, NJ (US); Suong NGUYEN, Princeton, NJ (US)

(73) Assignee: The Trustees of Princeton University, Princeton, NJ (US)

(21) Appl. No.: 18/028,278

(22) PCT Filed: Sep. 24, 2021
(86) PCT No.: PCT/US2021/051876

§ 371 Date: Mar. 24, 2023

Related U.S. Application Data
(60) Provisional application No. 63/083,573, filed on Sep. 25, 2020.

(51) Int. Cl. C01B 32/28; B01J 19/12

(52) U.S. Cl. CPC C01B 32/28; B01J 19/121; B01J 2219/12; B01J 2219/0879; B01J 2219/0892

(57) ABSTRACT

Disclosed is a method for wet chemical functionalization of single crystal diamond surfaces that involves gentle reaction conditions. This method enables a wide range of functional groups and molecules to be tethered to the diamond surface. The method is compatible with the formation of stable nitrogen vacancy centers with long spin coherence times within nanometers of the diamond surface.

### CROSS-REFERENCE TO RELATED APPLICATIONS

This application claims priority to U.S. Provisional Pat. App. No. 63/083,573, filed Sep. 25, 2020, which is incorporated by reference in its entirety herein.

### STATEMENT REGARDING FEDERALLY SPONSORED RESEARCH OR DEVELOPMENT

This invention was made with government support under Grant No. DE-SC0018978 awarded by the Department of Energy and Grant No. 1936118 awarded by the National Science Foundation. The government has certain rights in the invention.

### BACKGROUND

Color centers in diamond offer the possibility of performing nanoscale nuclear magnetic resonance (NMR) and electron paramagnetic resonance (EPR) spectroscopy of small ensembles and even individual biomolecules. Recent milestones based on nitrogen vacancy (NV) centers in diamond include detection of the nuclear spin noise from a single ubiquitin protein, the detection of single electron spin defects external to the diamond, and NMR spectroscopy of microscale volumes of liquid with 0.5 Hz spectral resolution. NV centers have also been used to map the precise location of up to 27 ¹³C nuclear spins at cryogenic temperatures. A key goal is to extend these techniques to perform biologically relevant spectroscopy on intact biomolecules.

One of the main outstanding challenges in pursuit of nanoscale magnetic resonance spectroscopy of biomolecules is the immobilization of target molecules within nanometers of a highly coherent, stable, NV qubit sensor. Immobilization is necessary because an untethered molecule would otherwise diffuse out of the detection volume within a few tens of microseconds.

The main approaches for diamond surface functionalization are not compatible with coherent shallow NV centers or intact biomolecules. Diamond surface functionalization often relies on plasma or high pressure/temperature conditions, which can lead to subsurface damage that destabilizes NV centers. Furthermore, such harsh conditions severely limit the scope of molecules that can be attached to the surface. Wet chemical methods would be advantageous for avoiding subsurface damage and attaching biomolecule targets, but previous attempts at wet chemical surface functionalization of nanodiamonds rely on functionalizing defect states at the diamond surface, and are thus not compatible with high quality, single crystal diamond surfaces. These high-quality surfaces are crucial for the fabrication of devices with high quality stable quantum sensors (NV centers with long spin coherence time and good charge state stability).

Thus, a technique to immobilize the target molecules within nanometers of a stable and highly coherent nitrogen vacancy (NV) qubit sensor is useful and desirable.

### SUMMARY

The disclosed approach is understood to be the first application of visible-light photoredox chemistry to selectively functionalize the diamond surface. This suite of wet chemical techniques enables gentle conditions for surface functionalization, avoiding harsh plasma chemistry or high pressure reaction conditions. Another component of the disclosed approach is the creation of a mixed surface termination with high coverage, consisting of a halide atom termination alternating with an amide functional group that can serve as “handles” for subsequent functionalization. Additionally, these surface chemistries are compatible with the production of high-quality nitrogen vacancy (NV) centers within 30 nanometers, preferably within 20 nanometers, more preferably within 10 nanometers, even more preferably within 5 nanometers, and most preferably within 3 nanometers of the surface, allowing for their use as nanoscale quantum sensors.

The method for preparing a surface of a diamond comprising an NV center for functionalization can generally be understood as follows. Initially, the diamond will comprise a hydrogen-terminated surface. An intermediate diamond surface, that is prepared for functionalization, can then be created via exposing the hydrogen-terminated surface to a reaction mixture, which comprises: (i) a hydrogen atom transfer (HAT) reagent and a fluorinating reagent, (ii) a HAT reagent and a nitrile reagent, (iii) an N-chloroamide that can act as both a HAT reagent and a chlorination reagent, (iv) an N-xanthylamide that can act as both a HAT reagent and a xanthylation reagent, and (v) a fluorinating reagent. With options (i) to (iv), a photochemical reaction of the hydrogen-terminated surface with a reaction mixture occurs by exposing the surface to the reaction mixture and irradiating the surface with a predetermined wavelength of light (such as at least one wavelength between 300 and 500 nm) for a period of time (such as between 12 and 48 hours). With option (v), no photochemical reaction is utilized.

As discussed, the reaction mixture will have one of five possible compositions. For the photochemical reactions, the reaction mixture will comprise: (a) a hydrogen atom transfer (HAT) reagent (such as tetra-n-butylammonium decatungstate) and a fluorinating reagent (such as N-fluorobenzenesulfonimide, 1-Chloromethyl-4-fluoro-1,4-diazoniabicyclo[2.2.2]octane bis(tetrafluoroborate), 1-Fluoro-4-methyl-1,4-diazoniabicyclo[2.2.2]octane bis(tetrafluoroborate), or a combination thereof), (b) a HAT reagent and an nitrile reagent (such as acetonitrile, 3,3,3-trifluoropropionitrile, or trichloroacetonitrile), (c) an N-chloroamide reagent (such as N-(tert-butyl)-N-chloro-3,5-bis(trifluoromethyl)benzamide), or (d) an N-xanthylamide reagent (such as N-(tert-butyl)-N-((ethoxycarbonothioyl)thio)-3,5-bis(trifluoromethyl)benzamide). For the non-photochemical reaction, the reaction mixture will comprise a fluorinating reagent (such as nitrosyl tetrafluoroborate).

Advantageously, the photochemical reaction is configured such that after the reaction, the surface of the diamond comprises a halide atom termination alternating with a molecule containing at least one functional group handle adapted for subsequent functionalization.

Once the surface is prepared, a functionalized surface can be created by attaching a molecule of interest to the intermediate surface, using known techniques, either during the time in which the surface is being exposed to the reaction mixture, or afterwards. Advantageously, the molecule of interest is attached to the intermediate surface via a diverse set of reactions, such as click reaction, cross metathesis reaction, acylation reaction, nucleophilic substitution reaction, and thiol-ene reaction. In some embodiments, the molecule of interest is attached to the intermediate surface via a click reaction wherein an azide-containing substrate of interest undergoes a cycloaddition reaction with a tethered alkyne moiety in a surface-bound amide to form a triazole product, where a catalyst (such as a copper catalyst) is present. In other embodiments, the molecule of interest is attached to the intermediate surface via a cross metathesis reaction wherein an olefin-bearing molecule of interest, in the presence of a Grubbs catalyst, undergoes a cross metathesis reaction with a tethered alkene group on an amide to form a cross-coupled product. Furthermore, hydrolysis reaction of the surface-bound amide groups can afford amine-terminated surface, which can subsequently undergo acylation reaction with a carboxylic acid-containing molecule of interest to covalently attach this molecule to the surface. In addition, molecule of interest is attached to the intermediate surface via a nucleophilic substitution reaction wherein an amine-containing molecule of interest undergoes substitution reaction with a tethered bromine group in a surface-bound amide to form the substituted product. Lastly, a molecule of interest is attached to the intermediate surface via a thiol-ene reaction wherein a thiol-containing molecule of interest undergoes thiol-ene reaction with a tethered alkene group in a surface-bound amide to form the thioether product.

Advantageously, the molecule of interest is a fluorophore or a biomolecule (preferably a peptide or protein).

Advantageously, the diamond comprises nitrogen vacancy (NV) centers within 20 nanometers, more advantageously within 10 nanometers, even more advantageously within 5 nanometers, and most advantageously within 3 nanometers of the intermediate surface.

Advantageously, the center of mass of the molecule of interest, after being attached to the intermediate surface, is within 20 nanometers, more advantageously within 10 nanometers, even more advantageously within 5 nanometers, and most advantageously within 3 nanometers of a nitrogen vacancy (NV) center.

According to some aspects of the present disclosure, some additional processing steps may occur. For example, in some embodiments, an ultrahigh purity diamond is provided, after which nitrogen ions are implanted into the diamond, and then the diamond is subjected to high temperature annealing to form NV centers prior to surface termination. The surface may then be hydrogen terminated through various known methods. If the end application requires coherent shallow NV centers, it may be useful to hydrogen terminate by annealing in forming gas.

Advantageously, it may be useful during the processing to degas the reaction mixture and backfilling with an inert gas a plurality of times, then add a dry nonaqueous solvent prior to any irradiation.

Advantageously, some post-functionalization steps may be useful. For example, in some embodiments, the method also includes isolating the functionalized diamond from the reaction mixture, and iteratively washing the surface with one or more solvents. The iterative washing step may comprise or consist of, e.g., heating the diamond in a first solvent, and then consecutively sonicating the diamond in a plurality of solvents.

Advantageously, some additional post-functionalization steps may be useful to stabilize the charge state of shallow NV centers such as annealing the sample to low temperatures under an atmosphere of oxygen, cleaning the sample with strong acids, or illuminating the sample with laser excitation.

This method allows the production of specific diamond surfaces. Advantageously, what is produced is a diamond comprising an NV center with Hahn echo coherence times ranging between a few and hundreds of microseconds and a surface functionalized with a molecule of interest (such as a biomolecule), where the center of mass of the molecule of interest is within, e.g., 20 nanometers, 10 nanometers, 5 nanometers, or 3 nanometers of the NV center.

### DESCRIPTION OF DRAWINGS

FIG. 1 is a flowchart illustrating steps of an embodiment of a disclosed method.

FIG. 2A is an illustrated flowchart of an embodiment of a disclosed method using a first approach (photochemical fluorination reaction approach).

FIG. 2B is an illustrated flowchart of an embodiment of a disclosed method using a second approach (photochemical reactions for the formation of C—F and C—N bonds).

FIG. 2C is an illustrated flowchart of an embodiment of a disclosed method using a third approach (photochemical chlorination reaction).

FIG. 2D is an illustrated flowchart of an embodiment of a disclosed method using a fourth approach (photochemical xanthylation reaction).

FIG. 2E is an illustrated flowchart of an embodiment of a disclosed method using a fifth approach (non-photochemical fluorination reaction).

FIG. 3A is an illustrated flowchart of an embodiment of a disclosed method for functionalizing the second approach using click reactions.

FIG. 3B is an illustrated flowchart of an embodiment of a disclosed method for functionalizing the second approach using cross metathesis reactions.

FIG. 3C is an illustrated flowchart of an embodiment of a disclosed method for functionalizing the second approach using amide coupling reactions.

FIG. 3D is an illustrated flowchart of an embodiment of a disclosed method for functionalizing the second approach using nucleophilic substitution reactions.

FIG. 3E is an illustrated flowchart of an embodiment of a disclosed method for functionalizing the second approach using thiol-ene reactions.

FIG. 3F is an illustrated flowchart of an embodiment of a disclosed method for functionalizing the first approach using defluorination followed by alkylation reactions.

FIG. 4 is a graph showing NV depth and coherence times, for two fluorinated samples treated according to the disclosed methods,

### DETAILED DESCRIPTION

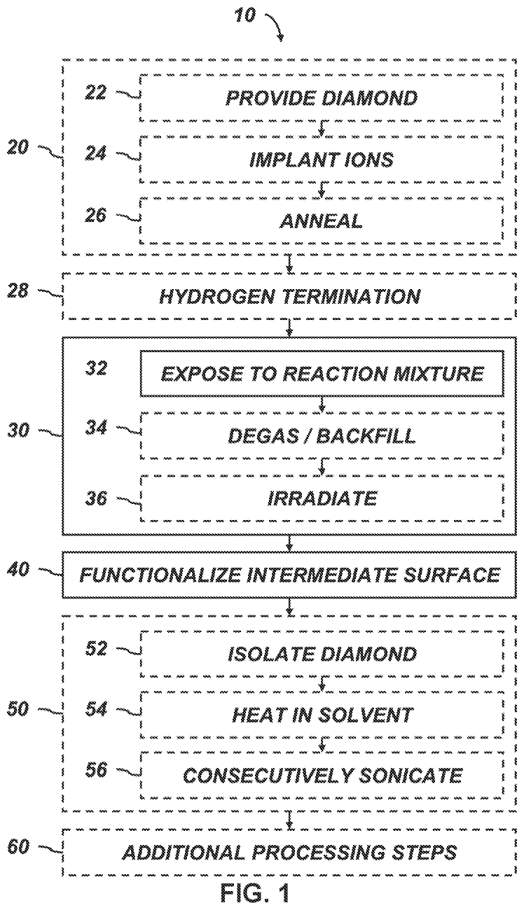

Referring to FIG. 1, the disclosed method (10) begins by optionally providing (20) a high-purity diamond. For samples that contain NV centers, after the diamonds are provided (22), nitrogen ions are implanted (24) and then the sample is subject to high temperature annealing (26) before all of the surface termination steps. Such steps are well-understood in the art (see, e.g., Nano Lett. 2014; 14(4) 1982-1986 and Phys. Rev. X 9, 031052).

Next, the surface is converted to a hydrogen-terminated surface (28). This may be accomplished via any appropriate technique. For example, as is known in the art, a diamond having polished, etched, and oxygen terminated surfaces can be converted to hydrogen terminated surfaces via application of a low damage plasma treatment. Additionally, it is known in the art that a CVD-grown diamond will have a hydrogen terminated surface after growth.

If end application requires coherent shallow NV centers, it may be advantageous to do the hydrogen termination via annealing to 800 C under forming gas (5% Hz, 95% Ar) for 1-5 days. Using this method, coherent NV centers were measured within 10 nm of a functionalized diamond surface, with dynamically decoupled coherence times exceeding 100 μs.

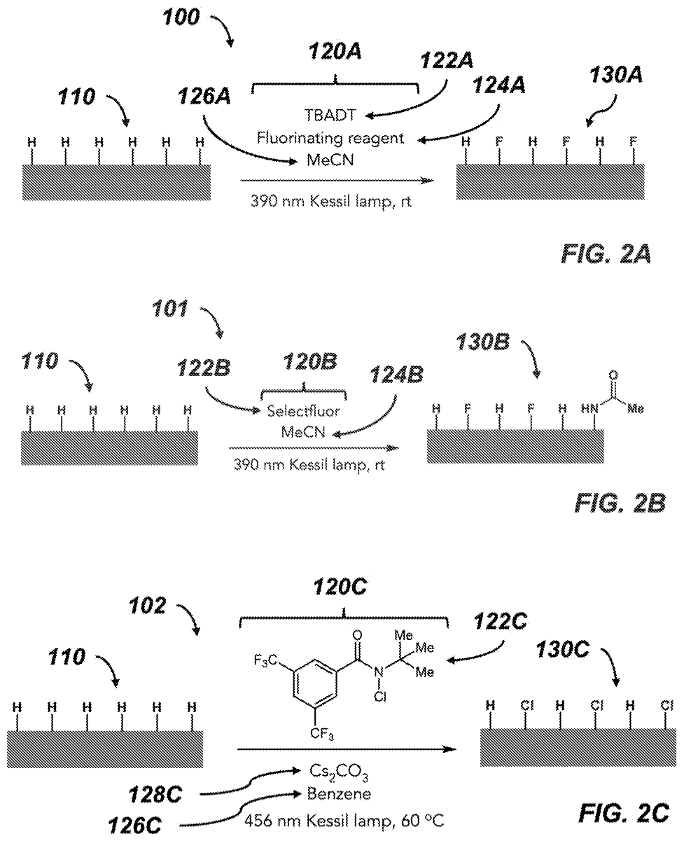

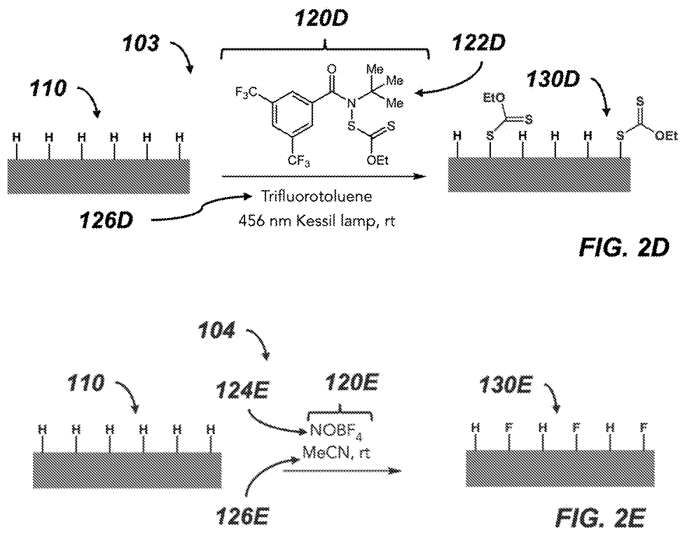

Once a diamond with a hydrogen-terminated surface is provided, an intermediate surface can be created (30). This is done via a photochemical reaction with a reaction mixture by exposing (32) the surface to the reaction mixture and irradiating (36) the surface with a predetermined wavelength of light for a period of time. There are two possible approaches here. The first approach uses a hydrogen atom transfer (HAT) reagent to abstract hydrogen atoms from C—H bonds on the surface, generating carbon-centered radicals. These radical intermediates can then be intercepted with a fluorinating reagent to form C—F bonds. The second approach uses the HAT reagent to abstract hydrogen atoms on the surface to generate carbon-centered radicals. These radicals can be either intercepted with a fluorinating reagent to form C—F bonds, or can be oxidized by selectfluor to afford a carbocation intermediate. This carbocation intermediate can react with a nitrile reagent via a Ritter mechanism to form C—N bonds. The third approach uses an N-chloroamide reagent that can serve as both HAT reagent and chlorination reagent to form C—Cl bonds. The fourth approach uses N-xanthylamide reagent that can serve as both HAT reagent and xanthylation reagent to form C—S bonds. The fifth approach uses the fluorinating reagent to form C—F bonds. These three possible approaches are illustrated in FIGS. 2A, 2B, 2C, 2D, and 2E, respectively.

Referring to FIG. 2A, the first approach (100) requires exposing a hydrogen-terminated diamond surface (110) to a reaction mixture (120A). In FIG. 2A, the reaction mixture (120A) contains a HAT reagent (122A) and a fluorinating reagent (124A), as well as a solvent (126A). After exposure to certain wavelengths of light (to activate the reaction)—here, 390 nm light from a Kessil lamp—for a period of time (here, 24 hours), the result is the creation of an intermediate surface (130A). In this case, the result is a partially fluorinated surface (e.g., alternating C—H and C—F bonds exist on the surface).

In preferred embodiments, the HAT reagent can be tetra-n-butylammonium decatung state, N-fluorobenzenesulfonimide (NFSI) or 1-Chloromethyl-4-fluoro-1,4-diazoniabicyclo[2.2.2]octane bis(tetrafluoroborate) (Selectfluor® reagent) or 1-Fluoro-4-methyl-1,4-diazoniabicyclo[2.2.2]octane bis(tetrafluoroborate) (Selectfluor® II reagent). Selectfluor®, Selectfluor® II, and NFSI reagents are available from Air Products and Chemicals. The HAT reagent will typically comprise between 1% and 25% w/w of the reaction mixture.

Any appropriate fluorinating reagent may be used. In preferred embodiments, the fluorinating reagent comprises NFSI, Selectfluor® reagent, 1-Fluoro-4-methyl-1,4-diazoniabicyclo[2.2.2]octanebis(tetrafluoroborate) (Selectfluor® II reagent), or a combination thereof. Selectfluor® II reagent is available from Air Products and Chemicals. The fluorinating reagent will typically comprise between 1% and 25% w/w of the reaction mixture.

In some embodiments, the HAT reagent is the same as the fluorinating reagent. In some embodiments, the HAT reagent is different from the fluorinating reagent.

The reaction mixture will generally also comprise a nonaqueous solvent, such as dry acetonitrile. The solvent will typically comprise between 30% and 99% w/w of the reaction mixture, such as between 75% and 90% w/w of the reaction mixture. In such embodiments, the method may also include a degassing step prior to the addition of the nonaqueous solvent. For example, in some embodiments, the method comprises degassing the HAT reagent/fluorinating reagent mixture and backfilling with an inert gas a plurality of times, then adding a dry nonaqueous solvent.

The reaction mixture may optionally comprise an additional component, such as sodium bicarbonate. If used, the additional components will typically comprise less than 1-5% w/w of the reaction mixture.

The hydrogen-terminated surface is then exposed to the reaction mixture and irradiated with a predetermined wavelength of light for a period of time, to allow the reaction to proceed. The predetermined wavelength will generally comprise at least one wavelength between 300 and 500 nm, and preferably one wavelength in the range of 370-460 nm (e.g., a near-UV wavelength). In some embodiments, the mixture is irradiated with a 390 nm wavelength of light. In other embodiments, the mixture is irradiated with a 456 nm wavelength of light. This irradiation will generally continue until the reaction is complete. In some embodiments, the irradiation continues for between 12 and 48 hours.

The reaction mixture is preferably kept at or around room temperature. In some embodiments, the reaction mixture is kept at between 20° C. and 25° C. throughout the entire period of time. In some embodiments, the reaction mixture is kept at approximately 25° C. throughout the entire period of time. In other embodiments, the reaction mixture is kept at approximately 65° C. throughout the entire period of time.

The reaction mixture is preferably kept at or around atmospheric pressure (for example, between 0.9 and 1.1 atm), or slightly above atmospheric pressure (for example, up to 2 atm).

### Example 1—First Approach: Photochemical Fluorination Reaction

A hydrogen-terminated diamond was added to a 2-dram vial along with a magnetic stir bar, tetra-n-butylammonium decatungstate (10 mg), and the fluorinating reagent (e.g., N-fluorobenzenesulfonimide (NFSI), Selectfluor® reagent, or Selectfluor® II reagent) (100 mg). The reaction mixture was degassed and backfilled with argon three times, after which 0.5 mL of dry acetonitrile was added. The reaction was then stirred at room temperature for 48 h under the irradiation of 390 nm purple Kessil lamps. The reaction temperature was maintained at approximately 25° C. over the course of the reaction.

Referring to FIG. 2B, the second approach (101) requires exposing a hydrogen-terminated diamond surface (110) to a reaction mixture (120B). In FIG. 2B, the reaction mixture contains a reagent that can be both a HAT reagent and an oxidant at the same time (122B) and a nitrile reagent (124B). After exposure to certain wavelengths of light (to activate the reaction) for a period of time, an intermediate surface (130B) is created. Here, it is seen that the surface is partially fluorinated (generally alternating C—H and C—F bonds), where some of the C—H bonds are replaced by an amide, where the nitrogen of the amide forms a C—N bond with the diamond surface.

Any nitrile reagent can be utilized here. For example, the nitrile may have the structure RCN, where R is a branched or linear alkyl group having any carbon chain lengths, where R is unsubstituted or substituted with one or more halogens. Non-limiting examples of nitriles include acetonitrile, 3,3,3-trifluoropropionitrile, and trichloroacetonitrile.

### Example 2—Second Approach: Photochemical C—N Bond Formation Reaction

A hydrogen-terminated diamond was added to a 2-dram vial along with a magnetic stir bar and Selectfluor (100 mg). The reaction mixture was degassed and backfilled with argon three times, after which 0.5 mL of dry acetonitrile was added. The reaction was then stirred at room temperature for 48 h under the irradiation of 390 nm purple Kessil lamps. The reaction temperature was maintained at approximately 25° C. over the course of the reaction.

### Example 3—Second Approach: Photochemical C—N Bond Formation Reaction

A hydrogen-terminated diamond was added to a 2-dram vial along with a magnetic stir bar and Selectfluor (60 mg). The reaction mixture was degassed and backfilled with argon three times, after which 0.5 mL of degassed 3,3,3-trifluoropropanenitrile was added. The reaction was then stirred at room temperature for 48 h under the irradiation of 390 nm purple Kessil lamps. The reaction temperature was maintained at approximately 25° C. over the course of the reaction.

### Example 4—Second Approach: Photochemical C—N Bond Formation Reaction

A hydrogen-terminated diamond was added to a 2-dram vial along with a magnetic stir bar and Selectfluor (60 mg). The reaction mixture was degassed and backfilled with argon three times, after which 0.5 mL of degassed trichloroacetonitrile was added. The reaction was then stirred at room temperature for 48 h under the irradiation of 390 nm purple Kessil lamps. The reaction temperature was maintained at approximately 25° C. over the course of the reaction.

Referring to FIG. 2C, the third approach (102) requires exposing a hydrogen-terminated diamond surface (110) to a reaction mixture (120C). In FIG. 2C, the reaction mixture contains an N-chloroamide reagent that can be both a HAT reagent and a chlorination reagent at the same time (122C), cesium carbonate (128C), and a solvent (126C). After exposure to certain wavelengths of light (to activate the reaction) for a period of time, an intermediate surface (130C) is created. In this case, the result is a partially chlorinated surface (e.g., alternating C—H and C—Cl bonds exist on the surface).

The N-chloroamide reagent used in the third approach is N-(tert-butyl)-N-chloro-3,5-bis(trifluoromethyl)benzamide.

### Example 5—Third Approach: Photochemical Chlorination Reaction

A hydrogen-terminated diamond was added to a 2-dram vial along with a magnetic stir bar, N-(tert-butyl)-N-chloro-3,5-bis(trifluoromethyl)benzamide (50 mg), and cesium carbonate (47 mg). The reaction mixture was degassed and backfilled with argon three times, after which 0.5 mL of dry benzene was added. The reaction was then stirred at room temperature for 24 h under the irradiation of 456 nm blue Kessil lamps. The reaction temperature was maintained at approximately 65° C. over the course of the reaction.

Referring to FIG. 2D the fourth approach (103) requires exposing a hydrogen-terminated diamond surface (110) to a reaction mixture (120D). In FIG. 2D, the reaction mixture contains an N-xanthylamide reagent that can be both a HAT reagent and a xanthylation reagent at the same time (122D), and a solvent (126D). After exposure to certain wavelengths of light (to activate the reaction) for a period of time, an intermediate surface (130D) is created. In this case, the result is a partially xanthylated surface, where some of the C—H bonds are replaced by a xanthate ester group, where the sulfur of the xanthate ester forms a C—S bond with the diamond surface.

The N-xanthylamide reagent used in the third approach is N-(tert-butyl)-N-((ethoxycarbonothioyl)thio)-3,5-bis(trifluoromethyl)benzamide.

### Example 6—Fourth Approach: Photochemical Xanthylation Reaction

A hydrogen-terminated diamond was added to a 2-dram vial along with a magnetic stir bar and N-(tert-butyl)-N-((ethoxycarbonothioyl)thio)-3,5-bis(trifluoromethyl)benzamide (170 mg). The reaction mixture was degassed and backfilled with argon three times, after which 0.5 mL of dry trifluorotoluene was added. The reaction was then stirred at room temperature for 24 h under the irradiation of 456 nm blue Kessil lamps. The reaction temperature was maintained at approximately 25° C. over the course of the reaction.

Referring to FIG. 2E, the fifth approach (104) requires exposing a hydrogen-terminated diamond surface (110) to a reaction mixture (120E). In FIG. 2E, the reaction mixture contains fluorinating reagent (124E) and a solvent (126E). After exposure to the reaction mixture for a period of time, an intermediate surface (130E) is created. Here, it is seen that the surface is partially fluorinated (generally alternating C—H and C—F bonds).

The fluorinating reagent used in the third approach is preferably a fluorinated nitrosating agent, such as nitrosyl tetrafluoroborate (NOBF₄).

### Example 7—Fifth Approach: Non-Photochemical Fluorination Reaction

A hydrogen-terminated diamond was added to a 2-dram vial along with a magnetic stir bar. The reaction vial was brought into the glove box and nitrosyl tetrafluoroborate (100 mg) was added. The vial was then sealed with a Teflon septum cap, removed from the glove box, and put under positive pressure of argon. Dry acetonitrile (0.5 mL) was added, and the reaction mixture was then stirred at room temperature for 24 h.

In all of the above-disclosed approaches, the resulting diamond (130A, 130B, 130C, 130D, and 130E) now comprises an intermediate surface that is partially or fully fluorinated, and/or amidated, or chlorinated, or xanthylated and is ready for functionalization. Preferably, the diamond comprises a stable NV center with long spin coherence times (Hahn echo spin coherence times in the range of a few to hundreds of microseconds) that is within 30 nanometers of the surface of the diamond, preferably within 20 nanometers, more preferably within 10 nanometers, even more preferably within 5 nanometers, and most preferably within 3 nanometers in a direction normal to the surface of the diamond.

Referring back to FIG. 1, after the intermediate surface is created (30), the method requires functionalizing (40) the intermediate surface.

The functionalization involves attaching a molecule of interest to the surface of the diamond. The molecule of interest may be any appropriate molecule of interest. In some embodiments, the molecules of interest are fluorophores, biomolecules, or a combination thereof.

As used herein, the term “biomolecule” refers to any molecule or biological component that can be produced by, or is present in, a biological organism. Biomolecules may include, but are not limited to, a lipid, a lipoprotein, a metabolome, a nucleic acid (DNA, RNA, microRNA, plasmid, single stranded nucleic acid, double stranded nucleic acid), an oligonucleotide, a peptide, a polypeptide, a polysaccharide, a protein, or a sugar, and may also include small molecules such as metabolites (including primary, metabolites, secondary metabolites, etc.) and other natural products, or any combination thereof. In some embodiments, the biomolecule is selected from the group of nucleic acids, peptides, polypeptides, and proteins.

In some embodiments, the molecules of interest are peptides or proteins.

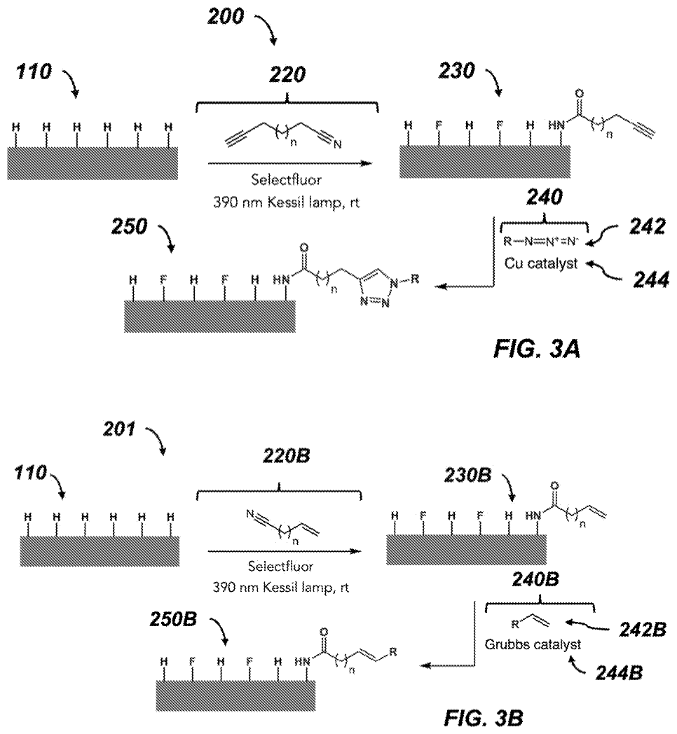

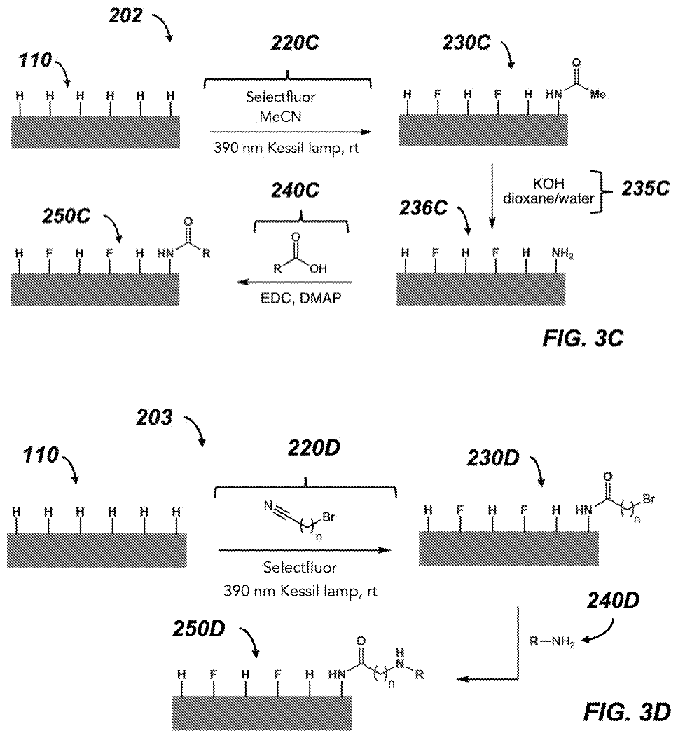

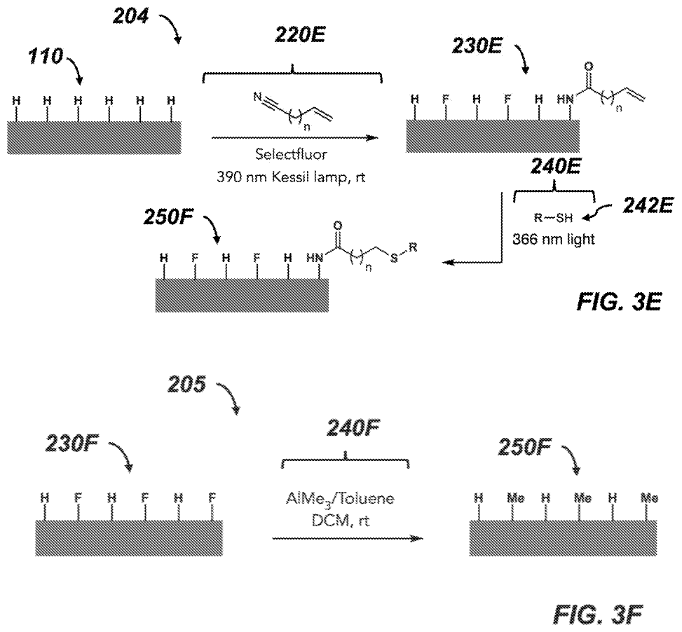

In some embodiments, such subsequent functionalization can be accomplished via click reactions, cross metathesis reactions, amide coupling reactions, nucleophilic substitution reaction, and thiol-ene reaction. That is, in order to attach various molecules of interest (e.g., fluorophores, peptides and proteins) to the diamond surface, one can use click chemistry (see, e.g., FIG. 3A), cross metathesis (see, e.g., FIG. 3B), amide coupling chemistry (see, e.g., FIG. 3C), nucleophilic substitution reaction (see, e.g., FIG. 3D), and thiol-ene reaction (see, e.g., FIG. 3E) for subsequent functionalization reactions.

A functional group necessary for these reactions (i.e., alkyne, alkene, amine, and bromide) should be present on a molecule attached to the surface during the photochemical reaction, or during a subsequent reaction.

Non-limiting examples of the functionalization of the surface are described below.

Referring to FIG. 3A, after the hydrogen-terminated surface (110) is exposed to the reaction mixture and the predetermined wavelengths of light (220), it results in an intermediate surface (230) where, e.g., an amide derivative with a terminal alkyne is covalently bound to the surface. In a subsequent reaction (240A), an azide-containing molecule of interest (“R”, 242A) will undergo a cycloaddition reaction with the alkyne on the amide to form the triazole product (with or without a copper catalyst (244A)), creating a functionalized surface (250A).

Similarly, referring to FIG. 3B, in some embodiments (201), after the hydrogen-terminated surface (110) is exposed to the reaction mixture and the predetermined wavelengths of light (220B), it results in an intermediate surface (230B) where an amide derivative with a terminal alkene is covalently bound to the surface. In the presence of a Grubbs catalyst (244B), an olefin-bearing molecule of interest (242B) will undergo a cross metathesis reaction (240B) with the tethered alkene group on the amide to furnish the cross-coupled product, resulting in a functionalized surface (250B).

In some embodiments, a second intermediate surface may be formed in the process of functionalizing the surface. Referring to FIG. 3C, in some embodiments (202), after the hydrogen-terminated surface (110) is exposed to the reaction mixture and the predetermined wavelengths of light (220C), it results in an intermediate surface (232) where an amide is covalently bound to the surface. A subsequent hydrolysis reaction (235C) with a base (e.g., potassium hydroxide) or an acid (e.g., hydrogen chloride) will generate another intermediate surface (236C) where an amine functional group is covalently bound to the surface. In the presence of 1-ethyl-3-(3-dimethylaminopropyl)carbodiimide hydrochloride (EDC) and 4-dimethylaminopyridine (DMAP) catalysts, a carboxylic acid-containing molecule of interest will undergo amide coupling reaction (240C) with amine group on the surface to furnish the amide-coupled product, resulting the functionalized surface (250C).

Referring to FIG. 3D, in some embodiments (203), after the hydrogen-terminated surface (110) is exposed to the reaction mixture and the predetermined wavelengths of light (220D), it results in an intermediate surface (230D) where an amide derivative with a terminal bromide is covalently bound to the surface. An amine-bearing molecule of interest (240D) will undergo a nucleophilic substitution reaction with the tethered bromide group on the amide to furnish the substituted product, resulting in a functionalized surface (250D).

Referring to FIG. 3E, in some embodiments (204), after the hydrogen-terminated surface (110) is exposed to the reaction mixture and the predetermined wavelengths of light (220E), it results in an intermediate surface (230E) where an amide derivative with a terminal alkene is covalently bound to the surface. Under irradiation of 366 nm light, a thiol-bearing molecule of interest (242E) will undergo a thiol-ene reaction (240E) with the tethered alkene group on the amide to furnish the addition product, resulting in a functionalized surface (250E).

Alternatively, referring to FIG. 3F, in some embodiments (205), after the hydrogen-terminated surface is converted to a partially fluorinated surface (230F), a subsequent reaction (240F) attaches a molecule of interest to the surface, resulting in a functionalized surface (250F). In FIG. 3, the surface is exposed to trimethylaluminum in the presence of toluene and dichloromethane, but other organoaluminum materials could be utilized. For example, exposing a partially or fully fluorinated diamond surface to a R₃Al, where R is a molecule of interest.

### Example 8: Separate Post-Fluorination Functionalization

A fluorinated-terminated diamond (obtained from previous photochemical fluorination reaction) was added to a 2-dram vial along with a magnetic stir bar. The reaction mixture was degassed and backfilled with argon three times, after which a solvent (here, 0.3 mL of dry dichloromethane) and a second reaction mixture comprising a functional group to be added to the surface (here, 0.1 mL of 2.0 M trimethylaluminum solution in toluene) were added. The reaction mixture was then stirred at room temperature for 24 h, resulting in a diamond surface having alternating C—H and C—CH₃ bonds.

Referring back to FIG. 1, after exposing the surface to the wavelength of light and functionalizing the intermediate surface, additional post-functionalization steps (50) may occur, such as steps for cleaning the diamond and preparing it for use. For example, in some embodiments, the diamond is isolated (52) from the reaction mixture, and subsequently washed with acetonitrile, water, acetone, and isopropanol. One then continues washing the surface iteratively with a variety of solvents selected to clean contaminants off the diamond surface while leaving the covalently bonded functional group intact. The cleaning procedure may begin with heating (54) the diamond(s) in dimethyl sulfoxide or acetone, followed by long consecutive sonications (56) in dimethyl sulfoxide, chloroform, distilled water, acetone, and isopropanol, in order. The temperature of the heating step as well as the specific sequence of solvents is adjusted based on the expected chemical stability of the functional group. Any organic solvent may be used for the washing step, and the order of solvents used may be changed. Advantageously, the solvents are selected so as to dissolve and remove any and all contamination that resulted from previous reactions, so the optimal solvents will necessarily depend upon what the previous reactions were. Preferably, between three and eight solvents are used. Preferably, isopropanol is the final solvent used, after which the sample is dried with nitrogen. In some embodiments, the temperature of the heating step is between 50° C. and 90° C.

In some instances, to observe charge-stable NV centers, some additional processing steps (60) may be necessary. For example, it may be necessary to remove residual hydrogen termination in a manner that does not destroy the functional group attached. Depending on the functional group, this could be accomplished in different ways. One option includes annealing the sample under an atmosphere of oxygen to low temperatures (between 100 and 460 C) for 1-10 hours. Another option includes refluxing the sample in a 1:1:1 volume mixture of sulfuric, perchloric, and nitric acids for 2 hours. Another option could be treating the sample with UV-ozone. An additional option could be illuminating the sample with a laser. It may be preferable to use a continuous-wave laser with a 532 nm, focused on the surface with a power ranging between tens of microwatts to a few milliwatts for a time period ranging from the order of seconds to a few hours. As understood by those of skill in the art, the above is a non-limiting example of laser parameters that may be useful. Other laser parameters may also be effective, that use different wavelengths, power, modes of operation (e.g., continuous-wave or pulsed), powers, time, etc.

One could also verify that physisorbed contaminants are removed using atomic force microscopy (AFM) imaging and X-ray photoelectron spectroscopy (XPS).

Finally, one can verify that a covalent bond formed between the surface groups and the diamond using near-edge X-ray absorption fine structure spectroscopy (NEXAFS) at a synchrotron facility.

Thus, the process results in a diamond comprising a charge-state stable NV center and a surface functionalized with a molecule of interest, the molecule of interest being close to an NV center. Preferably, the center of mass of the molecule of interest is located at least partially above the NV center, at a distance (in a direction normal to the surface of the diamond) that is within 30 nanometers, preferably within 20 nanometers, more preferably within 10 nanometers, even more preferably within 5 nanometers, and most preferably within 3 nanometers of the NV center.

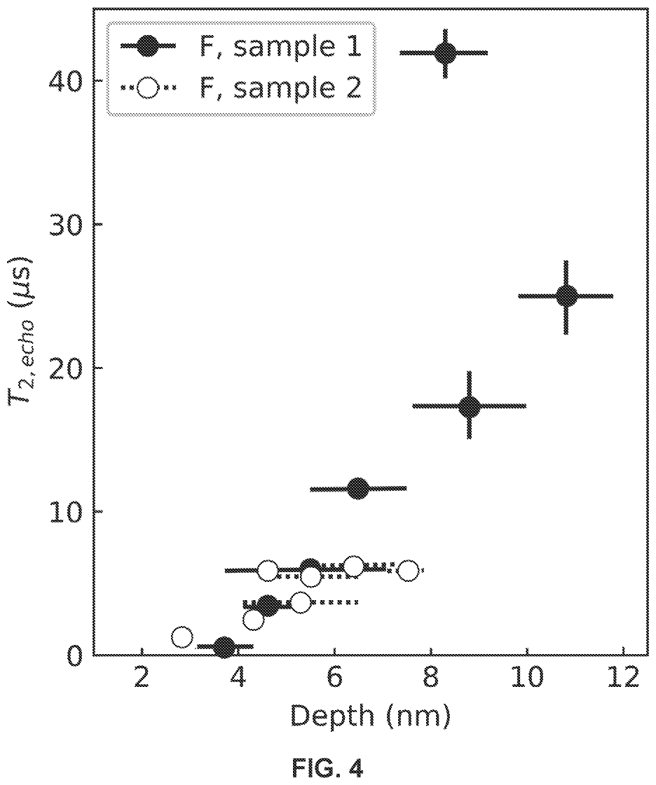

In addition to having charge-state stable NV centers, the disclosed method allows for NV centers to have reasonable coherence times at relatively shallow depths. Examples of measured depth and coherence of NV centers that were treated according to the above methodology can be seen in FIG. 4. In the first sample, there were NV centers with measured depths between just under 4 nm and just under 12 nm, with Hahn echo coherence times from a few μs to over 40 μs. In the second sample, there were NV centers with depths that ranged from under 3 nm to under 8 nm (i.e., between 2 nm and 8 nm), with Hahn echo coherence times between 1 and 10 μs. In FIG. 4, with some exceptions, the coherence times generally increased with increasing depth.

At depths of 3 nm, the Hahn echo coherence time for an NV center after using the disclosed method is preferably 1-5 μs. At depths of 5 nm, the Hahn echo coherence time for an NV center after using the disclosed method is preferably 4-10 μs. At depths of 10 nm, the Hahn echo coherence time for an NV center after using the disclosed method is preferably 18-50 μs. And at depths of 20 nm, the Hahn echo coherence time for an NV center after using the disclosed method is preferably 100-300 μs.

For each NV center at a depth of 20 nm or less, the Hahn echo coherence time is preferably between is between 1 μs and 300 μs after using the disclosed method. For each NV center at a depth of 10 nm or less, the Hahn echo coherence time is preferably between is between 1 μs and 50 μs after using the disclosed method. For each NV center at a depth of 5 nm or less, the Hahn echo coherence time is preferably between is between 1 μs and 10 μs v. And for each NV center at a depth of 3 nm or less, the Hahn echo coherence time is preferably between is between 1 μs and 5 μs after using the disclosed method.

The disclosed approach allows for the functionalization of the diamond surface to introduce sensing targets. For example, biomolecules could be placed on the surface within 30 nanometers, preferably within 20 nanometers, more preferably within 10 nanometers, even more preferably within 5 nanometers, and most preferably within 3 nanometers of an NV quantum sensor, allowing for the interrogation of a single biomolecule with nuclear magnetic resonance and magnetic resonance imaging techniques via the single quantum sensor.

Other possible uses for this surface chemistry include functionalization of the diamond surface for power electronics, electrochemistry, and cathodes for electron emission in vacuum.

Those skilled in the art will recognize or be able to ascertain using no more than routine experimentation many equivalents to the specific embodiments of the invention described herein. Such equivalents are intended to be encompassed by the following claims.

What is claimed is:

1. A method for functionalization a diamond surface, comprising:
providing a diamond with a hydrogen-terminated surface; and
creating a modified diamond surface via exposing the surface to a reaction mixture, wherein the reaction mixture comprises: (i) a hydrogen atom transfer (HAT) reagent and a fluorinating reagent, (ii) a HAT reagent and a nitrile reagent, (iii) an N-chloroamide reagent that can act as both a HAT reagent and a chlorination reagent, and (iv) an N-xanthylamide reagent that can act as both a HAT reagent and a xanthylation reagent, or (v) a fluorinating reagent.

2. The method according to claim 1, wherein creating the modified diamond surface comprises exposing the surface to a photochemical reaction of the hydrogen-terminated surface with a reaction mixture by exposing the surface to the reaction mixture and irradiating the surface with a predetermined wavelength of light for a period of time, the reaction mixture comprising (1) a hydrogen atom transfer (HAT) reagent and either a fluorinating reagent or a nitrile reagent, (2) an N-chloroamide reagent, or (3) an N-xanthylamide reagent.

3. The method according to claim 2, wherein the photochemical reaction is configured such that after the reaction, the surface of the diamond comprises a halide atom termination alternating with a molecule containing at least one functional group handle adapted for subsequent functionalization.

4. The method according to claim 2, further comprising creating a functionalized surface by attaching a molecule of interest to the intermediate surface.

5. The method according to claim 4, wherein the functionalized surface is created during the period of time.

6. The method according to claim 4, wherein the functionalized surface is created after the period of time.

7. The method according to claim 3, wherein the molecule of interest is attached to the intermediate surface via a click reaction, a cross metathesis reaction, an amide coupling reaction, a nucleophilic substitution reaction, and a thiol-ene reaction.

8. The method according to claim 3, wherein the molecule of interest is attached to the intermediate surface via a click reaction wherein an azide-containing substrate of interest undergoes a cycloaddition reaction with an alkyne attached to an amide to form a triazole product.

9. The method according to claim 8, wherein a copper catalyst is present.

10. The method according to claim 3, wherein the molecule of interest is attached to the intermediate surface via a cross metathesis reaction wherein an olefin-bearing molecule of interest, in the presence of a Grubbs catalyst, undergoes a cross metathesis reaction with a tethered alkene group on an amide to form a cross-coupled product.

11. The method according to claim 3, wherein the molecule of interest is attached to the intermediate surface via an acylation reaction wherein a carboxylic-bearing molecule of interest, in the presence of EDC and DMAP catalysts, undergoes an amide coupling reaction with amine groups on the surface formed upon hydrolysis reaction of the amide group to form acylated product.

12. The method according to claim 3, wherein the molecule of interest is attached to the intermediate surface via a thiol-ene reaction wherein a thiol-bearing molecule of interest undergoes a substitution reaction with a tethered alkene group on an amide to form an addition product.

13. The method according to claim 3, wherein the molecule of interest is attached to the intermediate surface via a nucleophilic substitution reaction wherein an amine-bearing molecule of interest undergoes substitution reaction with a tethered bromine group on an amide to form a substituted product.

14. The method according to claim 3, wherein the molecule of interest is a fluorophore, a peptide, or a protein.

15. The method according to claim 3, wherein the diamond comprises a nitrogen vacancy (NV) center within 20 nanometers of the surface.

16. The method according to claim 15, wherein the NV center is within 10 nanometers of the surface.

17. The method according to claim 16, wherein the NV center is within 5 nanometers of the surface.

18. The method according to claim 17, wherein the NV center is within 3 nanometers of the surface.

19. The method according to claim 3, wherein the molecule of interest is within 20 nanometers of a nitrogen vacancy (NV) center.

20. The method according to claim 19, wherein the molecule of interest is within 10 nanometers of the NV center.

21. The method according to claim 20, wherein the molecule of interest is within 5 nanometers of the NV center.

22. The method according to claim 21, wherein the molecule of interest is within 3 nanometers of the NV center.

23. The method according to claim 2, wherein the HAT reagent is 1-Chloromethyl-4-fluoro-1,4-diazoniabicyclo[2.2.2]octane bis(tetrafluoroborate).

24. The method according to claim 2, wherein the fluorinating reagent comprises N-fluorobenzenesulfonimide, 1-Chloromethyl-4-fluoro-1,4-diazoniabicyclo[2.2.2]octane bis(tetrafluoroborate), 1-Fluoro-4-methyl-1,4-diazoniabicyclo[2.2.2]octanebis(tetrafluoroborate), or a combination thereof.

25. The method according to claim 2, wherein the fluorinating reagent is also a HAT reagent.

26. The method according to claim 2, wherein the nitrile reagent is an acetonitrile, 3,3,3-trifluoropropionitrile, and trichloroacetonitrile.

27. The method according to claim 2, wherein the predetermined wavelength is between 300 and 500 nm, and the period of time is between 12 and 48 hours.

28. The method according to claim 2, wherein the N-chloroamide reagent is N-(tert-butyl)-N-chloro-3,5-bis(trifluoromethyl)benzamide.

29. The method according to claim 2, wherein the N-xanthylamide reagent is N-(tert-butyl)-N-((ethoxycarbonothioyl)thio)-3,5-bis(trifluoromethyl)benzamide.

30. The method according to claim 2, further comprising:
providing a high purity diamond substrate;
implanting nitrogen ions; and
subjecting the diamond to high temperature annealing to form NV centers prior to surface termination.

31. The method according to claim 30, wherein providing a diamond with a hydrogen-terminated surface comprises hydrogen terminating a surface of the diamond after subjecting the diamond to high temperature annealing to form NV centers.

32. The method according to claim 2, further comprising:
acid cleaning a functionalized surface of the diamond by refluxing in volume ratio 1:1:1 sulfuric nitric and perchloric acids for between 1 and 3 hours, or
annealing the functionalized surface of the diamond under an atmosphere of oxygen to temperatures <460° C. for a between 1 and 10 hours, or
illuminating the diamond with laser excitation.

33. The method according to claim 32, wherein the laser excitation utilizes a continuous wave 532 nm laser focused on the diamond surface with laser power between 10 microwatts to a 10 milliwatts for a duration between 1 second and 12 hours.

34. The method according to claim 2, further comprising degassing the reaction mixture and backfilling with an inert gas a plurality of times, then adding a dry nonaqueous solvent.

35. The method according to claim 2, further comprising:
isolating the diamond from the reaction mixture; and
iteratively washing the surface with one or more solvents.

36. The method according to claim 35, wherein the iterative washing comprises:
heating the diamond in a first solvent; and
consecutively sonicating the diamond in a plurality of solvents.

37. A diamond comprising an NV center with Hahn echo coherence times in the range of 1-300 microseconds and a surface functionalized with a molecule of interest, the molecule of interest being within 20 nanometers of the NV center.

38. The diamond according to claim 37, wherein the molecule of interest is within 10 nanometers of the NV center and the Hahn echo coherence times are in the range of 1-50 microseconds.

39. The method according to claim 37, wherein the molecule of interest is within 5 nanometers of the NV center and the Hahn echo coherence times are in the range of 1-10 microseconds.

40. The method according to claim 37, wherein the molecule of interest is within 3 nanometers of the NV center and the Hahn echo coherence times are in the range of 1-5 microseconds.

41. The diamond according to claim 37, wherein the Hahn echo coherence times are in the range of 100-300 microseconds.

42. The diamond according to claim 37, wherein the Hahn echo coherence times are in the range of 18-50 microseconds.

43. The diamond according to claim 37, wherein the Hahn echo coherence times are in the range of 4-10 microseconds.

44. The diamond according to claim 37, wherein the Hahn echo coherence times are in the range of 1-5 microseconds.
